(19) United States
(12) Patent Application Publication
(10) Pub. No.: US 2007/0015134 A1
(43) Pub. Date: Jan. 18, 2007

(54) Quantitative stabilized cell reference control products and methods

(75) Inventors: John A. Maples, Miami, FL (US); Rhonda A. Mills, Pembroke Pines, FL (US); Daniel J. Flagler, Miami, FL (US); Cecilia U. Smith, Lake Placid, FL (US); Francisco Estevez-Labori, Miami Beach, FL (US)

Correspondence Address: STERNE KESSLER GOLDSTEIN & FOX, P.L.L.C., 1100 NEW YORK AVENUE, N.W., WASHINGTON, DC, 20005, US

(73) Assignee: Beckman Coulter, Inc., Fullerton, CA (US)

(21) Appl. No.: 11/183,132

(22) Filed: Jul. 15, 2005

(51) Int. Cl. C12Q 1/00; G01N 33/567; G01N 33/53

(52) U.S. Cl. 435/4; 435/7.2; 977/900

(57) ABSTRACT

The invention relates to quantitative stabilized cell reference control products, their methods of manufacture, and their methods of use. Receptors, antigens, and ligands associated with stabilized cells may be quantitated by the methods of the present invention. Those quantitated stabilized cells may then be used to determine the receptor, antigen, and/or ligand density of unknown samples.

### FIELD OF THE INVENTION

The present invention pertains to the field of quantitative cell-based reference control products and methods for making and using the same.

### BACKGROUND

Quantitation of antigens and receptors provides the basis for immunoassay systems and it is useful for both clinical therapy and clinical diagnosis of many diseases. Quantitation also facilitates the understanding of biologic mechanisms.

A reliable reference control product with a known quantity of target antigen or receptor for comparison can be used to validate quantitation. Soluble antigens can be isolated and measured quantitatively thus allowing references and controls to be obtained. Such soluble antigens can be attached to polymeric surfaces such as beads, which can be assayed for the amount of antigen detectable on their surface. But cellular antigens, which are expressed on or within cells, are difficult to model with standards or reference materials attached to polymeric beads because the optical characteristics of cells and polymeric beads are so different. By way of example, these characteristics include refractive index and Mie light scatter differences. The surface of polymeric substances may also influence chemical interactions that differ from the interactions that occur on the surface of cells.

It is accordingly an object of the invention to provide a quantitative cellular reference control product. Further objects of the present invention are methods for making and using the quantitative cellular reference control products of the present invention.

### SUMMARY OF THE INVENTION

In accordance with the invention, quantitative reference control products are provided comprising a population of stabilized cells, wherein the stabilized cells have at least one epitope that has substantially retained its antibody-binding characteristic and wherein a quantitative epitope-binding value has been assigned to the stabilized cells. The quantitative reference control products of the present invention may comprise more than one population of stabilized cells.

The quantitative reference control products provided by the present invention may also comprise a population of stabilized cells, wherein the stabilized cells have at least one receptor that has substantially retained its binding characteristics and wherein a quantitative receptor-binding value has been assigned to the stabilized cells. The quantitative reference control products of the present invention may comprise more than one population of stabilized cells.

The present invention further provides methods of making a quantitative reference control product comprising: (a) selecting a population of cells; (b) stabilizing a quantity of the cells; (c) quantitating an epitope associated with the cells; and (d) assigning a quantitative epitope-binding value to the stabilized cells.

The present invention further provides methods of making a quantitative reference control product comprising: (a) selecting a population of cells; (b) stabilizing a quantity of the cells; (c) quantitating a receptor associated with the cells; and (d) assigning a quantitative receptor-binding value to the stabilized cells.

The present invention further provides methods of quantitating an epitope density of unknown cells comprising: (a) measuring epitope-binding signals of an aliquot of the unknown cells; (b) measuring epitope-binding signals of an aliquot of known reference cells with a predetermined epitope binding value; (c) comparing the epitope-binding signals of the aliquot of the unknown cells to the epitope-binding signal values of the known cells; and (d) assigning a quantitative epitope density to the unknown cells.

The present invention also provides methods of quantitating receptor density of unknown cells comprising: (a) measuring receptor-binding signals of an aliquot of the unknown cells; (b) measuring receptor-binding signals of an aliquot of known reference cells with a predetermined receptor value; (c) comparing the receptor-binding signals of the aliquot of the unknown cells to the receptor-binding signal values of the known cells; and (d) assigning a quantitative receptor density to the unknown cells.

The quantitative reference control products of the present invention may also be used as calibrator products.

It is understood that the epitope quantitating and receptor quantitating methods can be combined or separate.

Additional objects and advantages of the invention will be set forth in part in the description which follows, and in part may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate the invention and together with the description, serve to explain the principles of the invention.

### BRIEF DESCRIPTION OF THE FIGURES

FIG. 1 presents a comparison of the mean fluorescent intensity of live versus lyophilized cell lines using the cellular antigen CD19.

FIG. 2 presents a comparison of the mean fluorescent intensity of live versus lyophilized cell lines using the cellular antigen CD20.

FIG. 3 presents a comparison of the mean fluorescent intensity of live versus lyophilized cell lines using the cellular antigen CD45.

FIG. 4 presents a comparison of the expression of CD4 antigen on live normal donors against the expression of CD4 antigen on stabilized or lyophilized/reconstituted tissue culture cells using direct and indirect staining methods.

FIG. 5 presents a linear curve of the binding of FITC-labeled antibodies to BioCytex beads.

FIG. 6 presents a linear curve of the binding of PE-labeled antibodies to BioCytex beads.

FIG. 7 is a table that demonstrates the mathematical relationships from existing commercial quantitation beads (BioCytex or FCSC) to live and lyophilized cells.

FIG. 8 presents a CD20 PE antigen linear binding curve of ten different lyophilized cell lines.

FIG. 9 presents a CD19 FITC antigen linear binding curve often different lyophilized cell lines.

FIG. 10 is a table that shows that the relative antigen quantitation between the different cells (or ratio) is independent of the reference method and is associated with the assay method.

FIG. 11 shows the assignment of quantitative CD4 values to cells using a normal value of 50,000 CD4 sites.

FIG. 12 show the binding curves generated from fluorescently labeled CD20-PE on three different lyophilized cell lines.

FIG. 13 show the binding curves generated from radiolabeled CD20-PE on three different lyophilized cell lines.

### DESCRIPTION OF THE EMBODIMENTS

Reference will now be made in detail to the embodiments of the invention. The order of the steps presented herein represent only one embodiment of the present invention. It is understood that the order of the following steps is only exemplary and that the steps may be reordered.

Cell Stabilization

Cells are selected for stabilization. For example, human or animal cells may be used in the present invention.

Essentially all cells that contain an antigen or a receptor may be used. The antigen/receptor may be extracellular, intracellular, or soluble. Examples of cells suitable for use in the present invention include cell lines, such as RPMI 8226, Ball-1, MANCA, EB-3, Raji, FMH-59B, CA46, Daudi, Ramos, CEM-1, KG1a, Jurkat, MOLT-4, U937, HL-60, THP-1, etc. Those cell lines, and others, are well known in the literature.

Other cells that may be used in the present invention include fibroblast cells, mast cells, neutrophils, lymphocytes, basophils, eosinophils, monocytes, platelets, red blood cells, melanocytes, chondrocytes, keratinocytes, cardiomyocytes, skeletal muscle cells, epithelial cells, endothelial cells, tumor cells of all types (particularly melanoma, leukemic cells (including, for example, myeloid and lymphoid leukemic cells), and carcinomas (including those of the lung, breast, ovaries, colon, kidney, prostate, pancreas, and testes)), liver cells, kidney cells, and stem cells (such as haemopoetic, neural, skin, lung, kidney, liver, and myocyte stem cells), as well as precursors of the above cells.

Any stabilization protocol capable of preserving a receptor, antigen, or ligand associated with the stabilized cell may be used.

As used herein, a receptor, antigen, or ligand is associated with the stabilized cell where the association may be intracellular (within the cytoplasm, nucleus or other cellular organelles), membrane-bound (intracellular or on the cell surface), or a passively linked soluble plasma constituent. A passively linked soluble plasma constituent is a plasma constituent that may be associated with a membrane component. By way of example, TNFα is a passively linked soluble plasma constituent in that it associates with a membrane receptor, and that receptor may be stabilized with the TNFα bound to the receptor. Other passively linked soluble plasma constituents include cytokines, chemokines, lipopolysacharide binding protein (LPB), mannose-binding protein (MBP), pattern-recognition molecules (PRM), and others.

Stabilization protocols suitable for use in the present invention include lyophilization, aldehyde-fixation, methanol-fixation, methanol-acetone fixation, formaldehyde fixation, paraformaldehyde-gluteraldehyde fixation, paraformaldeyde-Triton fixation, paraformaldehyde-methanol fixation, ethylenglycol-bis-succinimidyl succinate (EGS) fixation, fixation with cross-linking agents, fixation by tanning methods, and stabilization using enzyme inhibition with or without cross-linking agents. Such protocols are generally known in the literature.

By way of example, a lyophilization protocol is set forth in U.S. Pat. No. 5,059,518 to Kortright, which protocol is incorporated herein by reference. A modification of the Kortright protocol is set forth in U.S. Pat. No. 5,861,311 to Maples at column 8, lines 50-67, which protocol is incorporated herein by reference.

A formaldehyde-ammonium salt stabilization protocol is described in U.S. Pat. No. 6,913,932 to Maples, which protocol is incorporated herein by reference.

It is understood that the binding characteristics of the cells of the present invention may be modified before or after stabilization. Because one embodiment of the present invention is directed to reference control products, it is understood that it may be desired to reduce or amplify the binding characteristics of the cells of the present invention. By modifying those properties, it is possible to obtain a control with a certain level of binding properties. Methods of modifying binding characteristics of the cells are generally known in the literature. For example, KG1a cells may be treated with iodoacetamide to modify CD34 expression. See, e.g., U.S. Pat. No. 5,861,311 to Maples at column 9, line 57 to column 10, line 6, which protocol is incorporated herein by reference. Blood cells may be CD4 depleted as disclosed in U.S. Pat. No. 5,763,204 to Maples using anti-CD4 conjugated magnetic beads. Column 4, line 40 to column 5, line 29, which protocol is incorporated by reference. Other such methods are generally known in the literature.

The selection of stabilization protocol may be influenced, at least in part, upon the antigen or receptor of interest. The selection of the stabilization protocol may also be influenced by the period of time that the stabilization protocol preserves the stabilized cells.

Because the present invention can be applied to many different cells and cell lines, no one stabilization protocol is preferred. Similarly, because there are many antigens or receptors of interest that may be stabilized by the methods of the present invention, it is understood that no one stabilization protocol is preferred to stabilize the antigen or receptor of interest.

The receptors, antigens, and ligands associated with the cells of the present invention should have substantially retained their binding characteristics upon cell stabilization. The receptors, antigens, and ligands have substantially retained their binding characteristic when they demonstrate specific binding. That binding may be enhanced or reduced in comparison to the native receptor, antigen, or ligand. However, if the loss or gain associated with the stabilization protocol is reproducible and reliable, it understood that is understood that those cells may be used in the present invention. A stabilization protocol is reproducible and reliable when the protocol yields consistent numbers of active binding sites.

As used herein, reference is made to antigens. It is understood that antigens contain one or more ligands. If a ligand is preserved such that it retains its specific binding then it is considered that the antigen is preserved. Thus, it is understood that the antigen has been preserved where the ligand of interest has been preserved, even if other ligands associated with the antigen were not preserved.

The Cells may be Stained with a Dye

The cells may be stained with a nanoparticle or a dye, such as a vital nuclear dye, a lipophilic dye, or a fluorescent dye. Staining with a dye allows the stained cells to be separated from unstained cells, using, for example, flow cytometric techniques. The staining may occur before, during, or after stabilization.

Vital nuclear dyes suitable for use in the present invention are known to those of skill in the art and are widely available through commercial suppliers such as Invitrogen (Carlsbad, Calif.), Calbiochem (San Diego, Calif.), and Exciton (Dayton, Ohio). Examples of such dyes include LDS-751 (available from Exciton) and bisbenzamide (Hoechst 33342 or H-33342, which are available from Calbiochem), SYTOX Blue, Chromomycin A3, Mithramycin, YOYO-1, SYTOX, Ethidium Bromide, 7-aminoactinomycin D, Acridine Orange, DRAQ-5, TOTO-1, TO-PRO-1, Thiazole Orange, and Propidium Iodide (PI), TOTO-3, and TO-PRO-3. One or more vital nuclear dyes may be used in the methods and products of the present invention.

Lipophilic dyes suitable for use in the present invention include DIO, DIR, FM4-64 (all of which are available from Molecular Probes).

Fluorescent dyes suitable for use in the present invention are known to those of skill in the art and are widely available through commercial suppliers such as Sigma (St. Louis, Mo.) and Synthegen (Houston, Tex.). Exemplary of the fluorescent dyes suitable for use in the present invention are fluorescein isothiocyanate (FITC), phycoerythrin (PE), TAMRA-dT, Carboxyfluorescein (FAM), Hexachlorofluorescein (HEX), Tetrachlorofluorescein (TET), JOE, LightCycler Red 640, Cascade Blue, Lucifer yellow, LightCycler Red 705, Allophycocyanin (APC), Carboxytetramethylrhodamine (TAMRA), Amidite, Oregon Green, BODIPY, Rhodamine, Carboxy-X-Rhodamine (ROX), Coumarin, Methoxycoumarin, Hydroxycoumarin, Aminocoumarin, Cy2, Cy3, Cy3.5, Cy5, Cy5.5, and Cy7. Other suitable fluorescent dyes include novel tandem dyes, such as PE-Cy5, PE-Cy5.5, PE-Cy7, APC-Cy7, APC-Cy7, and APC-Cy5.5. One or more fluorescent dyes may be used in the methods and products of the present invention.

Nanoparticles suitable for use in the present invention include Q-Dots (available from Quantum Dot Corp. (Hayward, Calif.)), BioPixels (available from BioCrystal (Westerville, Ohio)), and Evitags (Evident Technologies (Troy, N.Y.)). Those nanoparticles are small enough to enter the cell and they may be fluorescently labeled.

It is contemplated that more than one dye or nanoparticle may be used in the methods and stabilized cells of the present invention. It is further contemplated that vital nuclear dye, lipophilic dye, fluorescent dye, or nanoparticle may be used in combination.

Reconstitution of the Stabilized Cells

In the methods and products of the present invention, the stabilized cells may be reconstituted. Reconstitution buffers are generally known within the literature. By way of example, phosphate-buffered saline-based solutions may be used as the reconstitution buffer. Water may also be used to reconstitute cells. Other suitable buffers include Tris-based buffers, HEPES-based buffers, MOPS, MES, PIPES, bicine, citrate, carbonate, phosphate buffers, borate buffers, glycerine buffers, and standard balanced salt solutions (Ringers, Tyrode, Earle, Hanks, and Dulbeccos). The reconstitution buffer may contain proteins (such as albumins), sugars, divalent cations (Mg++, Mn++, Co++, Ni++, Ca++), chelating agents (EDTA, EGTA), enzyme inhibitors (aprotin, phenylmethlysulphonyl fluoride (PMSF), levamisole, or tetramizole), enzymes (DNAse, RNAse), and salts. Many reconstitution buffers are commercially available. Examples of such commercially available reconstitution buffers include ISOTON® and PBS, which are available from Beckman Coulter.

Quantitative Value Assignment

Detection systems that generate quantitative signals are suitable for use in the present invention. Such systems include fluorescence-based cellular analyzers. By way of example, those instruments include fluorescent instruments, flow cytometers, hematology flow analyzers, image analysis microscopy, and others. By way of specific example, the following hematology flow analyzers may be used in the present invention: FC 500, Epics® XL™M, and Altra (all of which are made by Beckman Coulter, Miami, Fla.). Hematology analyzers from Sysmex Corporation of America (Long Grove, Ill.), such as the SYSMEX-RAMI1 9500, SF-3000, and XE-21000, Abbott Laboratories (Abbott Park, Ill.), such as the CELL-DYN series of instruments, ABX Diagnostics (Montpellier Cedex, France), such as the PENTRA 60 and 120 Retic hematology analyzers, or Bayer Diagnostics (Tarrytown, N.Y.), such as the ADVIA 70 and 20 hematology analyzers, may be used in the present invention. Image analyzers suitable for use in the present invention include IC100 (Beckman Coulter, Miami, Fla.). Other systems suitable for use in the present invention include ELISA, chemiluminescent assays, and radioimmunoassay.

Quantitative values may be assigned to the cells or the stabilized and reconstituted cells using standardized reference methods. Under one embodiment of the present invention, several aliquots of the reconstituted cells, as representations of the remaining stabilized aliquots, are selected for antigen quantitation using, for example, a standardized reference method for that antigen.

Under another embodiment of the present invention, several aliquots of the reconstituted cells, as representations of the remaining stabilized aliquots, are selected for receptor quantitation using, for example, a substance that binds to the receptor site using, for example, a standardized reference method.

It is understood that antigen quantitation and receptor quantitation are not mutually exclusive and that they may be performed on the same cell. Alternatively, they may be performed simultaneously or sequentially on a mixture of at least two different cells.

It is understood that quantitation may be performed prior to stabilization. Cells that have a consistent and reproducible change in antigen (or receptor) density measurements upon stabilization may be quantitated prior to stabilization. It is understood that the change may be a loss, a gain, or even no change in quantitative values. Generally, cells that have a defined change in quantitative values are suitable to quantitation prior to stabilization. The quantitative value of the cells may then be assigned, with an appropriate correction factor, to the stabilized cells.

One standardized reference method suitable for use in the present invention is to correlate the mean fluorescent intensity of the antigen/receptor of interest to the mean fluorescent intensity values generated from commercially available quantitative beads. Such beads are available from BioCytex (Marseilles, France) and Bangs Laboratories, Inc. (Fishers, Ind.).

BioCytex produces a mixture of beads that are coated with a known and increasing number of mouse immunoglobulins (IgG) on their surface. Those varying numbers of immunoglobulins are presented as representing the number of antigen or receptors on cells. When a fluorescent labeled anti-mouse IgG is added, the fluorescent anti-mouse antibody attaches to the IgG bound to the bead. The fluorescent intensity measured (MFI) is considered to be directly related to the number of IgG molecules reported to occur on those particular beads.

A variation of the BioCytex product is the Quantum Simply Cellular (FCSC, Bangs Laboratories, Inc.). Those beads are labeled with a known amount of anti-mouse IgG. When fluorescent labeled mouse monoclonal antibody is added it attaches to the bead and the MFI is then related to the number of anti-IgG antibodies on the particular bead.

A difference between the use of stabilized cells and beads is that the cells present the same antigen or receptor that is being measured. The beads use a secondary comparison. Therefore, immunoglobulin/bead systems do not represent either the antigen/antibody binding that may occur on cell membranes or the receptor/ligand binding that may occur on cell membranes. Moreover, beads have a different physical and chemical presentation of the antigen of interest than, for example, on the cell surface membranes. In the case of the Quantum Simply Cellular (FCSC) beads, where the binding antibody (anti-mouse) is attached to the beads, the physical chemical nature of those attached antibodies does not present a representative model for cellular binding of any type. Cytometry 33:138-145 and 166-178 (1998).

Curves (mathematical relationships) from the existing commercial quantitation beads (BioCytex or FCSC) may be used to assign fluorescent values to the lyophilized cells according to the antigen/antibody system desired. This is only one example of a quantitative antigen assignment method to the stabilized/reconstituted cells.

Scatchard analysis or other statistical analyses of quantitation beads (e.g. BioCytex, FCSC) allows the stabilized cells to be assigned quantitative antigen values relative to whatever reference system is chosen.

Saturation binding experiments, competitive binding experiments, and kinetics experiments are all standard reference methods that can be used to establish quantitative values for stabilized cells. Those protocols are well known. Fluorescent and radioactive labeled antibodies can be used.

Direct antigen models are further standard reference methods that may be used to establish quantitative values of stabilized cells. For example, the literature reports that normal CD4 positive lymphocytes express approximately 50,000 CD4 antigen molecules on their surface. Cytometry 33(2):123-32 (1988). Normal samples may be labeled and analyzed. The average fluorescent value, corrected with a negative control, is taken to be equivalent to the accepted literature value of 50,000. Those values may then be used to assign CD4 antigen quantitation to stabilized cells. Similar reported values for other antigens/receptors can be used to assign quantitative values to stabilized cells, or they may be normalized to the CD4 values as a reference.

Quantitative Value Assignment to Unknown Samples

Stabilized assayed cells can be used to establish quantitative values on unknown samples. Stabilized assayed cells can be included in assays, either separately or in combination with unknown cells or with defined soluble-analyte containing media, to enable the quantitation of cellular or soluble antigens (or receptors), independent of the assay detection system involved.

The use of stabilized assayed cells and cell lines to establish a quantitation of antigen number and receptor reference on the cell surface can also be expanded to applications for analysis of soluble antigen in the media. Using standard competitive interference methods, the inhibition of antibody or receptor binding to the stabilized cells determines the amount of soluble antigen in the media.

The use of stabilized cells, such as lyophilized cells, which have been assayed by a standardized method for antigen detection, enables the use of any antigen detection method to be directly related to the unknown amount of antigen on the unknown cells and/or in media.

The antigens on stabilized cells are suitable for use as quantitative reference controls for the quantitation of antigens associated with separate unknown cells. The unknown cells may be identified and separated from the assayed stabilized cells based on unique fluorescent labels contained in the stabilized cells and/or unique optical or physical characteristics of the stabilized cells, e.g., size and/or unique cellular markers.

One application determines the amount of antigen on aliquots of stabilized cells that have been labeled with a nuclear fluorescent dye. Those cells would be used as an antigen reference for the assay of unknown samples, such as patient samples. As one example of doing the assay, the stabilized cells, with a predetermined amount of antigen, are reconstituted and assayed separately or in combination with unknown cells or unknown media. The assay of the antigen expression on the unknown cells or media is performed at the same time and in relation to the detection of the known amount of antigen on the stabilized cells. The antigen detection method can be the same for both the unknown and stabilized assayed cells.

Under one embodiment, the method of assay is the use of fluorescent labeled monoclonal antibodies by flow cytometry. Several different stabilized assayed cells, each of which express different levels of antigen and having different detection characteristics to separate them from the unknown cells, may be used in the assay.

A value is assigned to the stabilized cells in terms of the antigen or receptor density for the cell population or populations identified in the stabilized reconstituted samples. This value is related to the quantitation derived using a standard reference method. This antigen or receptor density value may be applied to the remaining lyophilized cell sample population(s). These cells are referred to as “known cells.”

Reconstituted cells, “known cells,” having the assigned reference value for antigen or receptor density, are then assayed in the presence of cells that are unknown in antigen or receptor density. The “unknown cells” and “known cells” can be assayed separately or mixed together.

In one embodiment, the “unknown cells” are assayed in association with more than one sample of “known cells.” Each “more than one” sample generally have a different antigen/receptor density and different means of separating them from each other and from the unknown cells. This could be done from example on the basis of some combinations of fluorescence and/or antigen and/or receptor and/or light scatter characteristics.

The “known cells” may be assayed using the same reagents and reagent system used for the assay of the “unknown cell(s)” sample. The measurement system used in the assay may be applied to both the “known” and “unknown cells” assayed using the same reagents.

The measurement value of the “unknown” sample is compared with the measurement value of the one or more “known” cells, the “known cells” having one or more predetermined antigen or receptor quantitation value(s).

A mathematical relationship may be used to assign a quantitative antigen or receptor quantitation value to the “unknown cell(s).” A ratio of the assigned quantitative value measurement to the unknown quantitative measurement would be one example. Another example would be to use the different assigned values of the several stabilized “known” cells and plot a relationship between these values and then use this relationship to determine the “unknown cell(s)” antigen or receptor quantitation value.

The results from the standardized reference method may be analyzed by one or more statistical methods. Such methods include Scatchard analysis, Lineweaver-Burke plots, Eadie-Hofstee plots, double-reciprocal plots, Homes plot, semilogrithmic plots (Klotz, (1982) Science, 217, 1247), Hill equation/coefficient, linear regression, and non-linear regression. Such statistical methods allow the stabilized cells to be assigned quantitative values relative to whatever reference system is chosen.

Under one embodiment, a quantitative value is initially assigned to a master lot. A master lot is a lot from which future quantitative values may be assigned. For example, a master lot may be a seed lot from which further lots are grown. However, it is understood that a master lot need not be a parent lot. From a master lot, subsequent lots may be directly assigned their quantitative values.

Thus the invention of the use of stabilized cells to establish direct antigen and/or receptor values provides improvements and advances in therapeutics and diagnostics.

### Reference Control Products

Stabilized, assayed cells provide for the establishment of reference control products.

The reference control products of the present invention may have a predetermined quantitative value for antigen density. Alternatively, the reference control products of the present invention may have a predetermined quantitative value for receptor density. The reference control products of the present invention may contain a plurality of stabilized assayed cells. The plurality of stabilized, assayed cells may have predetermined quantitative values for antigen densities, predetermined quantitative values for receptor densities, or combinations of antigen and receptor densities.

In one embodiment, at least two different cell types are stabilized and supplied as a reference control product. The at least two different cells may be packaged into a single vial or they may be separately packaged.

In another embodiment, at least three different cells are stabilized and supplied as a reference control product. The at least three different cells may be packaged into a single vial or they may be separately packaged.

In another embodiment, at least one of stabilized cells has been stabilized by lyophilization.

When a reference control is supplied with at least two different cells, it is possible to prepare a regression curve of their quantitative values for subsequent assignment to unknown samples.

A reconstitution buffer may be supplied with the reference control product of the present invention. In at least one embodiment, the stabilized cells may be liquid, ready-to-use.

Calibrator Products

In one embodiment of the present invention, the stabilized assayed cells are supplied as a calibrator. As used herein, a calibrator is a stabilized, assayed cell of the present invention that can be used to determine the deviation from a standard so as to ascertain the proper correction factor for an instrument. Thus, a calibrator will generally have a tighter assignment of the assayed quantitative value than a reference control would have. The assignment of that assayed quantitative value is arrived at during the validation process of preparing the calibrator product. That value, which is assigned a tighter standard deviation than a reference control product, is dependent on the instrument to be calibrated and the inherent characteristics of that calibrator. The validation processes for preparing calibrators are generally known.

Calibrators, such as this embodiment of the present invention, are used by technicians to verify the working parameters of an instrument. Thus, the assigned values of a calibrator are used to confirm that the instrument is functioning properly. If the values from the instrument are different than the assigned values of the calibrator, the technician adjusts the instrument to achieve the “correct” values.

### EXAMPLES

### Example 1

### Comparison of MFI for Live Versus Stabilized Cells

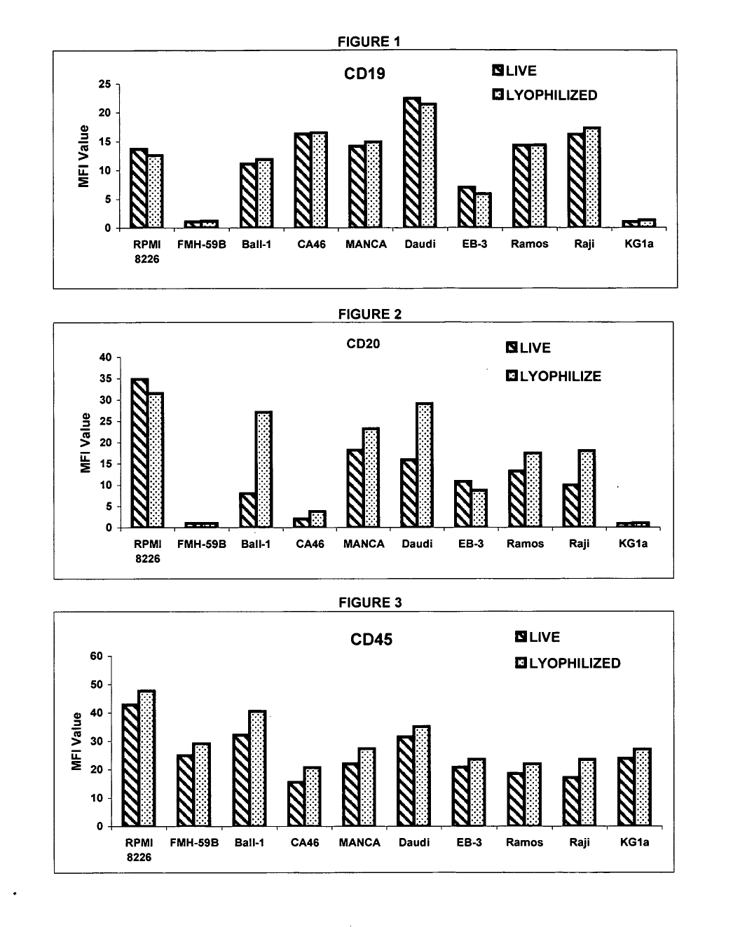

In the following example, cell lines RPMI 8226, Ball-1, MANCA, EB-3, Raji, FMH-59B, CA46, Daudi, Ramos, and KG1a were compared for their cellular antigens CD19, CD20, and CD45, using a direct stain with fluorescent labeled antibodies. See FIGS. 1, 2, and 3. The fluorescent intensity values on the y-axis (MFI) compares the live (not lyophilized) cells to the same cells, which were lyophilized using the protocol set forth in the Kortright patent (U.S. Pat. No. 5,059,518) and then reconstituted in PBS buffer. The antigen expression on the cell lines changed due to the lyophilization/reconstitution process. The CD20 antigen expression was different on the lyophilized vs. the live cells, sometimes higher and sometimes lower. Thus, the value assignment was made on the reconstituted lyophilized cells.

### Example 2

### Differences in Assay Expression of the CD4 Antigen on Live Versus Stabilized Cells

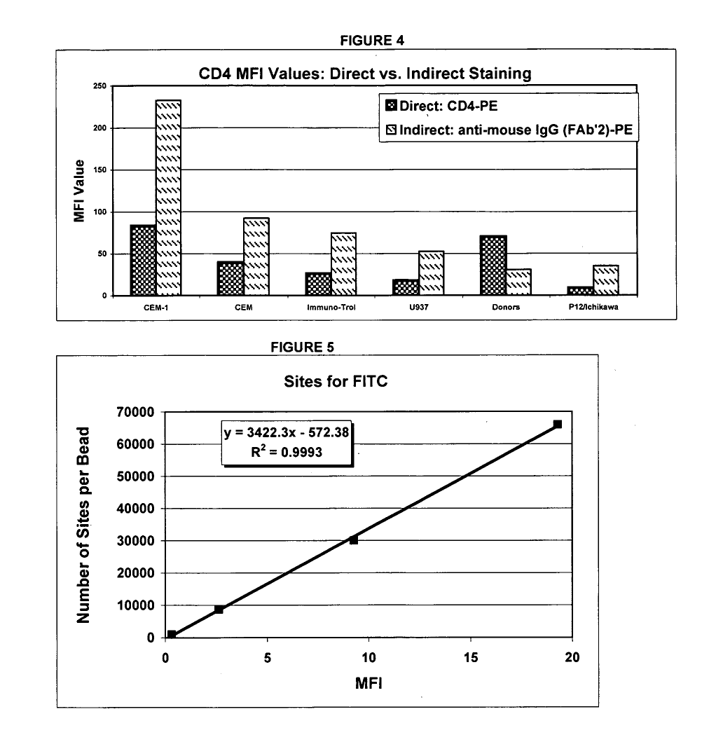

The expression of CD4 antigen on live white blood cells from normal donors (Donors) was compared against the expression of CD4 antigen on stabilized (Immuno-Trol™) and lyophilized/reconstituted tissue culture cells (CEM-1, U937, and P12/Ichikawa). FIG. 4.

The tissue culture cells (CEM-1, U937, and P12/Ichikawa) were lyophilized according to the Kortright protocol and reconstituted in PBS.

The donor cells, stabilized cells and the lyophilized/reconstituted tissue culture cells were stained directly using a CD4-PE fluorescent label. Separate aliquots of those cells were stained indirectly with unlabeled CD4 followed by incubation with an anti-mouse IgG (FAb′²) antibody-PE fluorescent label.

Differences in assay expression were seen in the cells labeled directly with CD4-PE fluorescent label vs. indirectly with anti-mouse IgG (FAb′²) antibody-PE. In the stabilized and lyophilized/reconstituted samples, the MFI values were reduced with the direct CD4-PE label relative to the MFI values obtained with the indirect anti-mouse IgG (FAb′²) antibody-PE label. In the normal, non-lyophilized donors the MFI values of the cells labeled directly with CD4-PE were higher than the MFI values obtained with the indirect anti-mouse IgG (FAb′²) antibody-PE label. This indicates that the expression of the CD4 on lyophilized cells was different from the CD4 expression on non-lyophilized cells.

### Example 3

### Generation of Linear Curves Using BioCytex Beads

BioCytex beads coated with different known concentrations of IgG on their surface (960, 8600, 30000, and 66000 sites per bead) were incubated separately with FITC and phycoerythrin labeled antibodies.

The CD19-FITC labeled and CD20-PE labeled antibodies were obtained commercially (Beckman Coulter, Inc., Fullerton, Calif.).

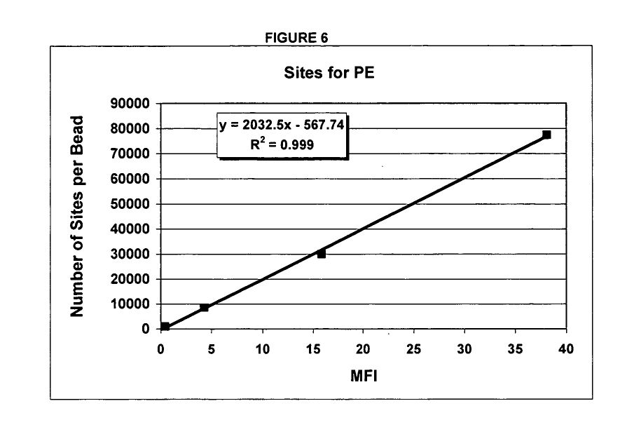

The MFI for the beads was measured using an FC 500. The MFI was then plotted against the number of sites per bead. See FIG. 5 and FIG. 6. Linearity was demonstrated for both FITC labeled and PE labeled antibodies using the BioCytex beads.

### Example 4

### Assignment of Fluorescent Values to Lyophilized Cells Using the Linear Curves Derived from BioCytex Beads

Cell lines RPMI 8226, FMH-59B, Ball-1, CA46, MANCA, Daudi, EB-3, Ramos, Raji, and KG1a were lyophilized according to the Kortright protocol.

Aliquots of the live and lyophilized cells were incubated with CD19, CD20, and CD45 antibodies. The antibodies were purchased from Beckman Coulter, Inc. (Fullerton, Calif.). The cells were analyzed by an FC 500 to obtain their MFI.

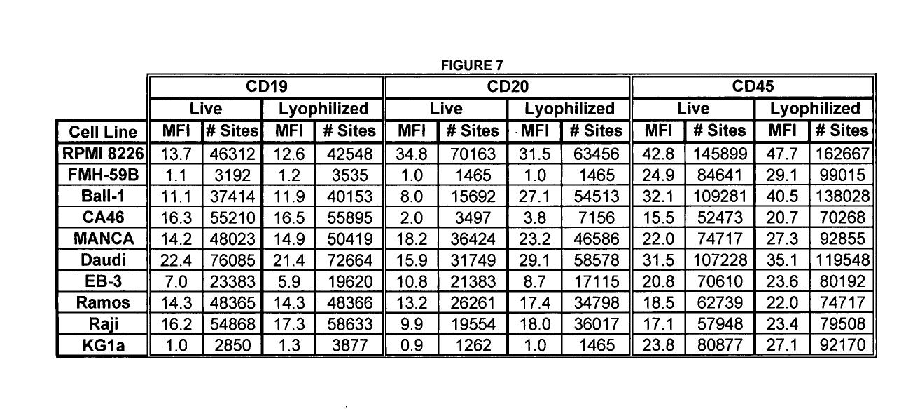

Mathematical relationships from existing commercial quantitation beads (BioCytex or FCSC) were used to assign fluorescent values to the lyophilized cells according to the antigen/antibody system desired. This is seen in the FIG. 7. The ten lyophilized-reconstituted cell lines were assigned quantitative antigen values based on the BioCytex beads (BioCytex Sites). As indicated in FIG. 7, antigen expression was different between live and lyophilized cells.

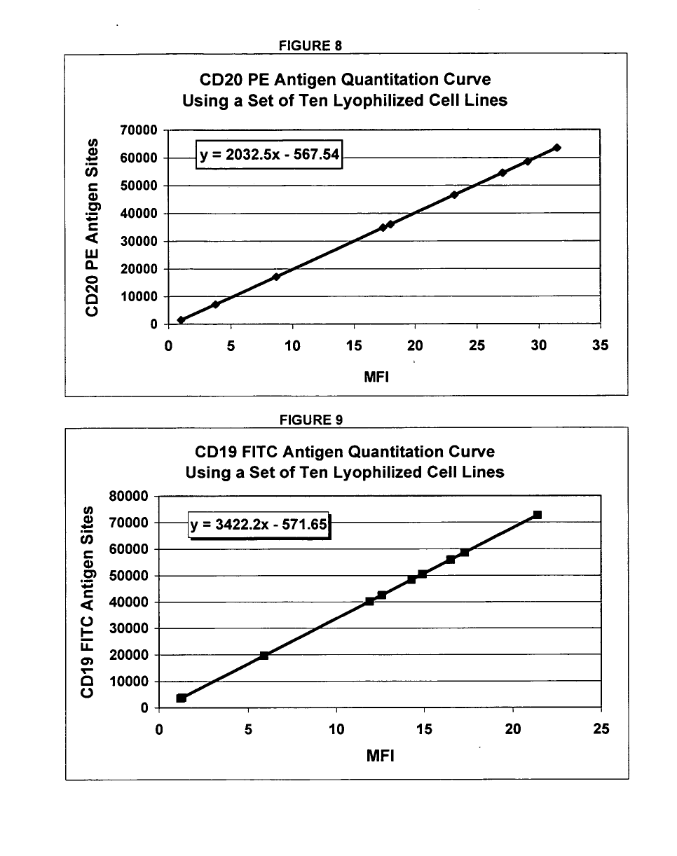

Scatchard analysis or other variations of the use of quantitation beads (e.g. BioCytex, FCSC) allows the lyophilized cells to be assigned quantitative antigen values relative to whatever reference system is chosen. The regression curves produced in FIG. 5 and FIG. 6 were based on the fluorescence obtained from the BioCytex bead quantitative assignments. Ten lyophilized cell lines were assigned quantitative antigen expression values (FIG. 8 and FIG. 9) for CD20-PE and CD19-FITC based on the regression curves obtained from that bead set.

Whereas the lyophilized cells present the same antigen or receptor that is being measured, the beads use a secondary comparison. The actual quantitative value of the antigen assigned to each lyophilized cell type was relative to whatever reference method is used (e.g. BioCytex Sites, FCSC site, Scatchard analysis).

### Example 5

### Relative Antigen Quantitation Between Different Cells (or Ratio) is Independent of the Reference Method

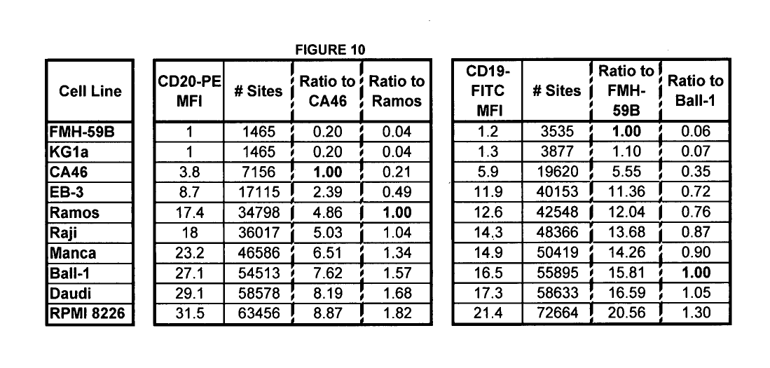

Once a quantitative value is assigned, the relative antigen quantitation between the different cells (or ratio) is independent of the reference method and is associated with the assay method. This is shown in FIG. 10 using the various cell lines.

The CD20 ratio relationship was based on the cell lines CA46 and Ramos, for example. The CD19 ratio relationship was based on the cell lines FMH-59B and Ball-1, for example. Thus, once an antigen quantitation value is associated with the lyophilized cells, antigen assay systems can be used and the assay value of the “known” lyophilized cells compared with the “unknown” cells and a value assigned to the “unknown.” The use of lyophilized cells is a direct model for the antigen or receptor expression on unknown cells. Bead-based methods use secondary models or representations. Ratio measurements using lyophilized cells or the use of multiple lyophilized cell lines, having different quantitative expression of receptor or antigen, can be used to directly establish quantitative antigen or receptor values on unknown samples using any assay system.

### Example 6

### Establishing Quantitative Values Using Lyophilized Cells Based on Direct Antigen Models

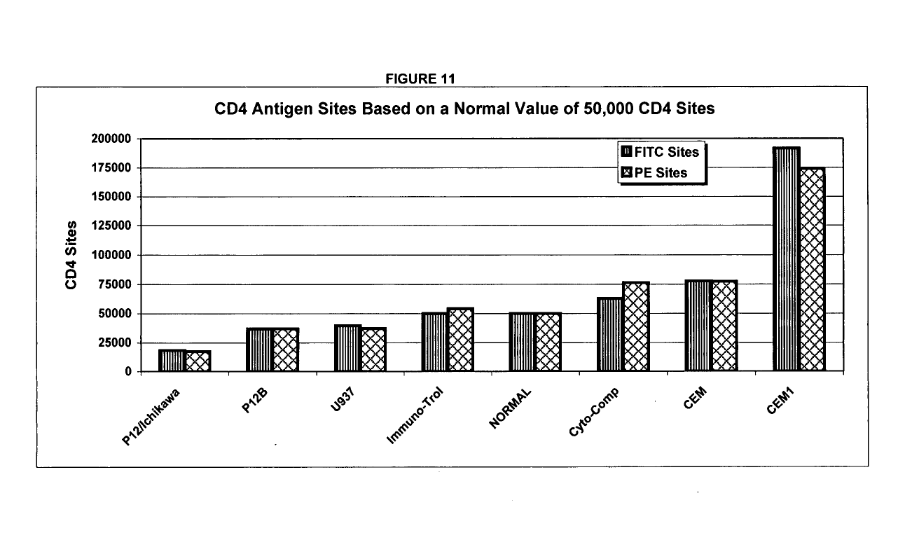

FIG. 11 shows another application for establishing quantitative values. This application assigned quantitative values to lyophilized cells using direct antigen models. Normal CD4 positive lymphocytes express approximately 50,000 CD4 antigen molecules on their surface. Cytometry 33(2):123-32 (1998). Normal samples were labeled with CD4-FITC and analyzed. The same normal samples were separately labeled with CD4-PE and analyzed. The average fluorescent value, corrected using a negative isotype control, was taken to be equivalent to the accepted literature value of 50,000 sites. Those values were used to assign CD4 antigen quantitation to the lyophilized cell lines P12/Ichikawa, P12B, U937, CEM-1, the lyophilized normal lymphocytes (Cyto-Comp™ Cells), and liquid preserved normal cells (Immuno-Trol™).

The preserved normal cells (Immuno-Trol™) quantitatively express the CD4 antigen similar to the normal donor expression of CD4 for both CD4-FITC and CD4-PE. The lyophilized normal lymphocytes (Cyto-Comp) have a higher expression of CD4. The lyophilized cell lines have similar CD4-FITC and CD4-PE with the exception of CEM-1 where the CD4-FITC was higher than the CD4-PE. The CD4-PE monoclonal antibody is greater than 2.5 times the size of CD4-FITC monoclonal antibody. This difference in CD4 labeling may be due to difference in size of the labels causing steric hindrance at high antigen expression. Such steric hindrance is important to quantitation in both cells and on beads, but only the antigen expression on lyophilized cells models the actual physical 3-D presentation that would occur on unknown cells. Attached immunoglobulin or anti-immunoglobulin to a bead does not model similar antigen expression. Using beads, this steric hindrance artifact on the cell surface at high antigen density could be missed.

### Example 7

### Measurement of Antibody Binding Sites on Lyophilized Cell Lines Radio labeling of Antibodies

The CD20 antibody was radioactively labeled by the Chloramine-T method. See Hunter and Greenwood, Nature 194:495-496 (1962); Greenwood et al., J.S. Biochemical Journal 89: 114-123 (1963). This method preserves the activity of the antibody.

Absorbance at 280 nm (A280) was used to determine the concentration of the unlabeled and labeled antibody. An extinction coefficient of 1.0 was used to convert A280 values to mg/mL. Standards were used to verify that the spectrophotometer was calibrated. The specific radioactivity was approximately 66 Ci/mmol.

Radiolabeled antibody was diluted to a stock concentration of 0.1 mg/mL and then serially diluted in PBS with 0.1% BSA. Antibody dilutions were used at 200 μL per test and were added to 100 μL of cell suspension during the binding assay.

### Suspension and Enumeration of Cells

Lyophilized cells (RPMI 8226, Ramos, and CA46), which were lyophilized according to the Kortright protocol, were resuspended in 1.0 mL of PBS with 0.1% BSA. Cell concentration was measured using the Flow-Count™ fluorospheres (from Beckman Coulter). To 100 μL of cell suspension, 1 mL of PBS was added. 100 μL of Flow-Count was added, vortexed, and counted on an FC 500 analyzer. The calibration factor, 1.041, for the lot of Flow-Count was applied. The cell number of stock suspension was adjusted to 2.5 million per mL. For each binding assay, 0.1 mL (250,000 cells) was used.

### Binding Assay

Radiolabeled antibody dilutions were aliquoted into microfuge tubes and the cell suspension was then aliquoted to each tube and incubated for at least one hour at room temperature.

PBS/BSA was added to each tube and the tubes were centrifuged. The first supernatant was removed to a separate labeled tube for gamma counting. The cells were washed twice more with PBS/BSA.

After resuspension to 1.0 mL with PBS/BSA, 100 μL of Flow-Count were added to each tube. An aliquot was removed to a separate tube for gamma counting. The remainder of the supernatant was analyzed by flow cytometry.

### Flow Cytometry

Cells were run on a FC500 cytometer using FS and SS parameters. A gate was drawn around the cell population and another around the bead population (CAL). At least 10,000 Flow-Count events were collected.

### Equilibrium

In this example, incubation was performed overnight at 4° C., which permits binding to reach equilibrium. The radioactivity of the supernatant was measured after centrifugation. Since the amount of bound ligand is likely greater than 10%, the initial supernatant was saved for gamma counting to obtain a measure of the free concentration.

### Non-Specific Binding

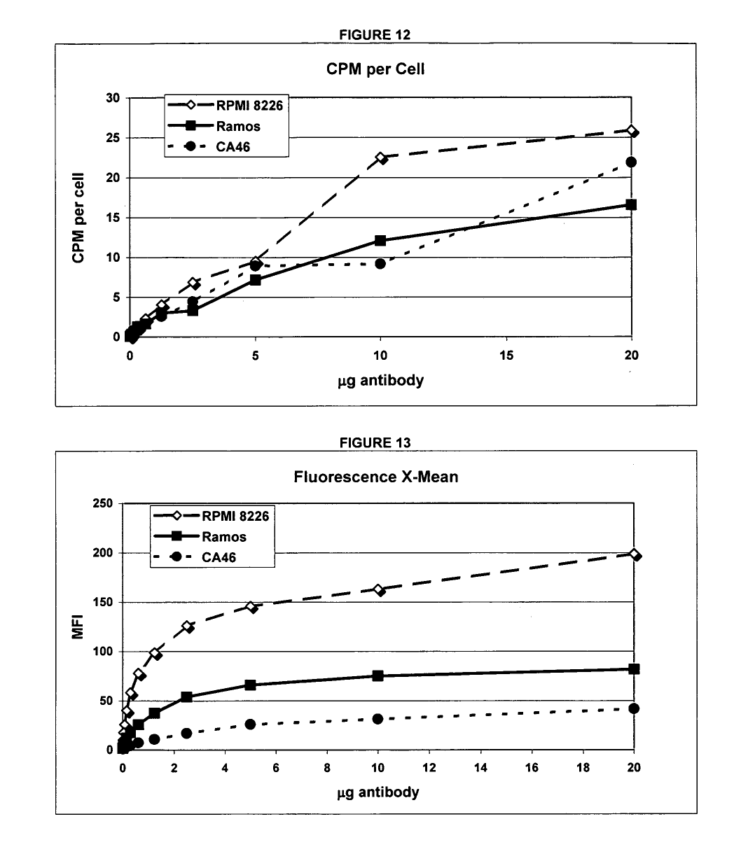

FIGS. 12 and 13 show the binding curves generated from fluorescently and radioactively labeled CD20-PE, respectively. Those figures show what appears to be non-specific binding for the RPMI 8226 cell line.

In an attempt to measure non-specific binding, cold antibody (40 μg per 100,000 cells) was incubated with the cells before addition of the radioactively labeled CD20-PE. From this experiment, the level of non-specific binding for the RPMI 8226 cells was approximately 8% of the total amount of radioactivity measured or 0.5 cpm per cell. This result was artificially low and may be due to the low number of cells counted in the assay or the low amount of cold antibody utilized.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A quantitative reference control product comprising a population of stabilized cells, wherein the stabilized cells have at least one epitope that has substantially retained its binding characteristic and wherein a quantitative epitope-binding value has been assigned to the stabilized cells.

2. The quantitative reference control product of claim 1, further comprising a second population of stabilized cells, wherein each population of stabilized cells has at least one epitope that has substantially retained its binding characteristic and wherein a quantitative epitope-binding value has been assigned to each population of stabilized cells.

3. The quantitative reference control product of claim 1, wherein the stabilized cells further contain a dye or a nanoparticle.

4. The quantitative reference control product of claim 3, wherein the dye is a vital nuclear dye, a lipophilic dye, or a fluorescent dye.

5. The quantitative reference control product of claim 1, wherein the epitope is an epitope on a cellular antigen or a soluble antigen.

6. The quantitative reference control product of claim 1, wherein the stabilized cells are lyophilized cells, aldehyde-fixed cells, or methanol-fixed cells.

7. The quantitative reference control product of claim 1, wherein the quantitative reference control product is a quantitative calibrator product.

8. The quantitative reference control product of claim 2, further comprising a third population of stabilized cells, wherein each population of stabilized cells has at least one epitope that has substantially retained its binding characteristic and wherein a quantitative epitope-binding value has been assigned to each population of stabilized cells.

9. A quantitative reference control product comprising a population of stabilized cells, wherein the stabilized cells have at least one receptor that has substantially retained its binding characteristic and wherein a quantitative receptor density value has been assigned to the stabilized cells.

10. The quantitative reference control product of claim 9, wherein the stabilized cells further contains a nanoparticle or a dye.

11. The quantitative reference control product of claim 9, wherein the quantitative reference control product is a quantitative calibrator product.

12. The quantitative reference control product of claim 9, further comprising a second population of stabilized cells, wherein each population of stabilized cells has at least one receptor that has substantially retained its binding characteristic and wherein a quantitative receptor density value has been assigned to each population of stabilized cells.

13. A method of making a quantitative reference control product comprising:
(a) selecting a population of cells;
(b) stabilizing a quantity of the cells;
(c) quantitating an epitope or a receptor associated with the cells; and
(d) assigning a quantitative value to the stabilized cells.

14. The method of claim 13, wherein the quantitation of the associated epitope is performed on the stabilized cells.

15. The method of claim 13, further comprising:
(e) staining a quantity of the cells with a dye or a nanoparticle.

16. A quantitative reference control product made according to the method of claim 13.

17. A quantitative calibrator product made according to the method of claim 13.

18. A method of quantitating epitope density of unknown cells comprising:
(a) measuring epitope-binding signals of an aliquot of the unknown cells;
(b) measuring epitope-binding signals of an aliquot of known reference cells with a predetermined epitope binding value;
(c) comparing the epitope-binding signals of the aliquot of the unknown cells to the epitope-binding signal values of the known cells; and
(d) assigning a quantitative epitope density to the unknown cells.

19. The method of claim 18, wherein the assignment is based upon a quantitative value determined from a master lot.

20. The method of quantitating epitope density of unknown cells of claim 18, further comprising aliquots of two or more distinct known reference cells, each with a predetermined epitope value, and using a mathematical relationship to assign a quantitative epitope value to the unknown cells.

21. A method of quantitating receptor density of unknown cells comprising:
(a) measuring receptor-binding signals of an aliquot of the unknown cells;
(b) measuring receptor-binding signals of an aliquot of known reference cells with a predetermined receptor value;
(c) comparing the receptor-binding signals of the aliquot of the unknown cells to the receptor-binding signal values of the known cells; and
(d) assigning a quantitative receptor density to the unknown cells.

22. The method of quantitating receptor density of unknown cells of claim 21, further comprising aliquots of two or more distinct known reference cells, each with a predetermined receptor value, and using a mathematical relationship to assign a quantitative receptor value to the unknown cells.
